(19) United States
(12) Patent Application Publication
(10) Pub. No.: US 2025/0152407 A1
(43) Pub. Date: May 15, 2025

(54) CONDOM HOLDER

(71) Applicant: Vadim Mihajlovich GERASIMENKO, Saint-Petersburg (RU)

(72) Inventor: Vadim Mihajlovich GERASIMENKO, Saint-Petersburg (RU)

(21) Appl. No.: 18/835,617

(22) PCT Filed: Feb. 2, 2022
(86) PCT No.: PCT/RU2022/000028

§ 371 Date: Aug. 2, 2024

(51) Int. Cl. A61F 6/04

(52) U.S. Cl. CPC A61F 6/04; A61F 2006/047

(57) ABSTRACT

A condom holder includes a base with means for gripping the condom ring, and locking devices. The locking devices are made in the form of latches with a stopper. The base is made detachable. The means for gripping the condom ring are hook-shaped. The base is provided with handles for holding with fingers. The technical result is to ensure the fixation of a condom when putting it on (as the condom ring is unwound), as well as during transportation and storage in the individual packing.

### TECHNICAL FIELD

The invention relates to the field of hygiene and human health, namely to the individual packing of condoms used as a means of contraception and prevention of infection transmission, in particular, to a condom holder designed to facilitate putting on a condom, avoiding its contamination and tearing when putting on.

### PRIOR ART

Known from the prior art is a packing for a condom, comprising two sheets of flexible film, soldered together at their ends to form a cavity for placing a condom in a rolled-up state (patent RU2562052, publ. 10.09.2015). One of the flexible sheets is provided with a line of least resistance (opening line), made in the form of a circle. The said sheet is provided with a tab for gripping with fingers and easy opening. After opening, the user removes the condom and puts it on the erect penis with hands.

Such packing does not contain any means to facilitate putting a condom on the erect penis and does not provide the required level of hygiene, since the user removes and puts on the condom with hands, which may not be clean enough. In addition, the need for direct contact between the fingers and a condom results to its tearing due to damage by nails, jewelry, dry, rough skin of the fingers, etc., and also as a result of rubbing off the lubricant with hands (the condom gets dry, which results to its tearing). All of the above is aggravated by tense emotional state of the user, which does not allow the condom to be positioned accurately on the erect penis without excessive stretching.

The closest prior art is a condom holder disclosed in patent RU2343880, publ. 20.01.2009, comprising grippers for fixing the edge of a condom and a connection between these grippers, at least partially being elastic, at least partially defining a space in which the condom can be placed; the holder being adapted to change the shape of said connection between two configurations: a first configuration in which said space is reduced, and a second configuration in which said space is expanded. In this case, in the first configuration at least part of said connection is strained and tends to transform into the second configuration, and in the second configuration said strain at least partially is released. The grippers are hook-shaped. The holder is provided with a handle designed for holding by the user's hand when putting on the condom.

The disadvantage of the known technical solution, chosen as a prototype, is the unreliable holding of a condom in the folded state (during transportation and storage, when it is not yet in use), as well as when putting on the erect penis. This is due to the fact that the device is held by the user with only one hand, which is often not the dominant (i.e., a hand with less developed motor skills), and thus does not provide a desired degree of effort when putting on the condom. In this case, putting on with one hand does not allow precise positioning of the device in such a manner that the trajectory of the device is parallel to the axis of the penis (so that the plane of the condom ring is perpendicular to the planes passing through the axis of the penis). All of the above results to the fact that the ring of the rolled condom jumps off the grippers at the beginning of putting on, making the device no longer usable, or in the process of putting on as the thickness of the condom ring decreases, which also makes it impossible to continue using the device and the condom.

### SUMMARY OF THE INVENTION

The technical problem is the need to create a condom holder that ensures the condom fixation in it during putting on (as the condom ring is unwound), as well as during transportation and storage in the individual packing.

The technical result is achieved due to the fact that a condom holder, including a base with means for gripping the condom ring, according to the invention, contains locking devices.

In particular embodiments of the invention, a locking device is made in the form of a latch with a stopper; the base is made detachable; means for gripping the condom ring are hook-shaped; the base is provided with handles for holding with fingers.

The presence of locking devices in the condom holder makes it possible to roll out (unwind) the condom evenly, without distortions, and to quickly and easily separate parts of the base from the condom ring without touching the condom with fingers. In addition, locking devices ensure permanent fixation of the condom in the individual packing during transportation and storage, even when the individual packing was exposed to impacts that result to the base breaking (along the lines of temporary connections), and therefore, allow the use of a condom with a broken base.

During the development of the claimed condom holder, tests confirming the achievement of the claimed technical result were carried out. To this end, 100 condoms of various sizes were put on a device simulating the erect penis (glass bulb). Half of the samples (50 pcs.) were put on in a traditional manner, i.e. rolled out by hands, and the other half via the use of the claimed holder. Careful putting on the samples of the first group (traditional method of putting on) took about 13-20 seconds; when trying to shorten the time of putting on, rolling out the condom was hampered by the distortion of the condom ring, as a result of which some of the condoms were torn (43%). The samples of the second group (putting on via the use of the claimed device) were put on in 2-3 seconds, without wrinkling and tearing, without jumping off the locking devices. Thus, the design features of the holder ensure that the condom is fixed in it during putting on (as the condom ring is unwound), as well as during transportation and storage in the individual packing.

### BRIEF DESCRIPTION OF THE FIGURES

The claimed invention is accompanied by drawings, where

FIG. 1 illustrates a top view of a condom holder (two latches are open),

FIG. 2 illustrates a bottom view (two latches are open),

FIG. 3 illustrates a general view of a condom holder (axonometric projection, one latch is closed),

FIG. 4 illustrates an enlarged view of a latch, FIG. 5 illustrates a top view of a condom holder during use (the condom is not shown).

### DETAILED DESCRIPTION OF THE INVENTION

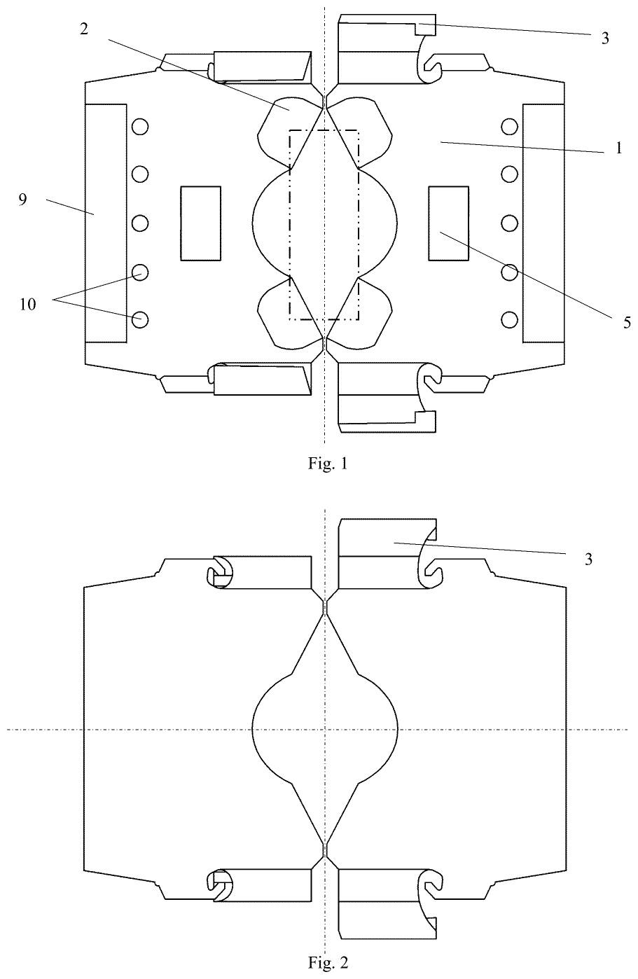

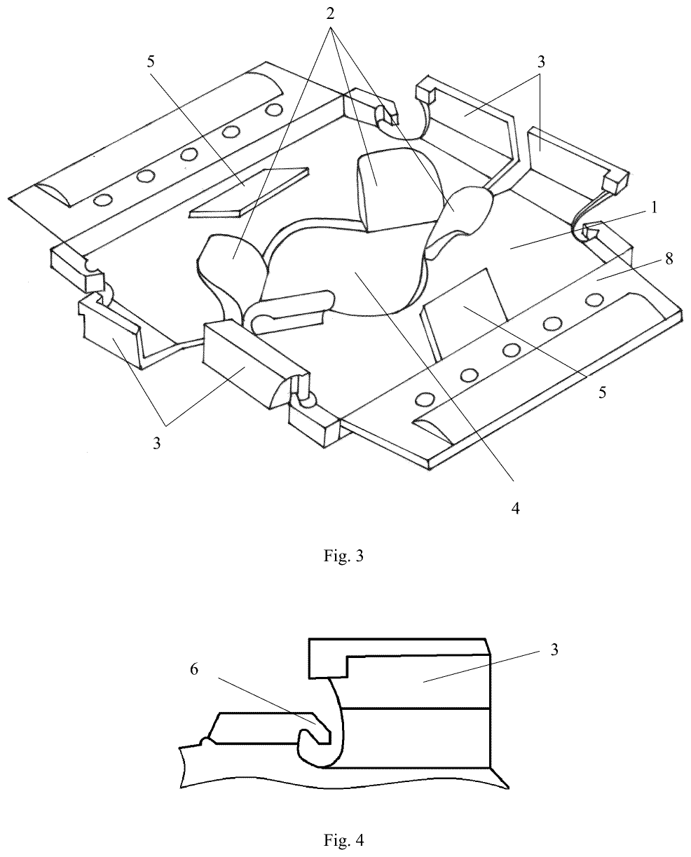

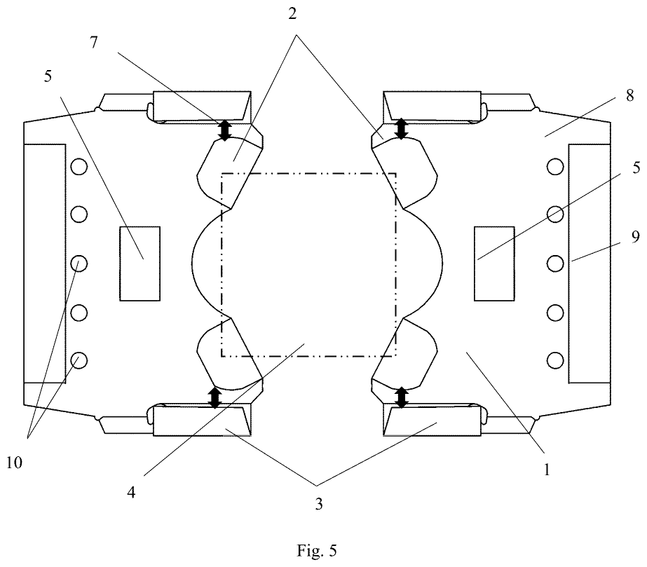

The condom holder is a base 1 on which the means 2 for gripping the condom ring, i.e. the condom in a folded state (the condom is not shown), and locking devices 3 are located (FIG. 1-5).

The base 1 is made detachable, preferably with the possibility of dividing into two parts. In this case, to ensure the safety of the condom during storage and transportation in the individual packing, parts of the base are interconnected by temporary connections that break when the base is stretched in different directions (for example, by jumpers) or at a slight bending.

The middle part of the base 1 has a hole 4, along the perimeter of which a condom ring (nimbus) is placed.

The base 1 is provided with means 2 for gripping the condom ring, located along the perimeter of the hole. The gripping means 2 can be made of any shape that ensures the orientation of the condom relative to the hole 4 and adapted to gently affect the condom when putting it on (to stretch the condom when parts of the base are spread in different directions). In the preferred embodiment, these means 2 are hook-shaped, for example, as shown in FIGS. 1-5, in a way that their ends are directed outward from the geometric center of the base 1 (the grippers are located on the inside of the placed condom ring). In this case, the bending radius of the gripping means 2 may be slightly greater than or equal to the thickness of the rolled condom ring (but not less, in order to avoid damage to the integrity of the condom). The number of means 2 for gripping a condom can be different and depends on the size of the base 1 and the placed condom, preferably at least four. In particular embodiments, the holder can be provided with additional gripping means 5, the hook-shaped ends of which are directed towards the geometric center of the base 1. Such gripping means 5 prevent the condom from jumping off the main gripping means 2, for example, at a strong bending of the packing (and, accordingly, the base) during transportation and storage.

The locking devices 3 are located on the base 1 outside the condom ring. The design of the locking devices 3 may be different. In the preferred embodiment of the device, they can be made in the form of latches with a stopper 6, namely in the form of hook-shaped elements pivotally fixed on the base, the fixation of which in the closed position is ensured by stoppers 6 in the form of hooks, with the formation of gaps 7 (spacing) between the end section of each hook-shaped element and the end section of the corresponding gripping means 2 (gaps 7 are shown with double arrows in figures). In particular embodiments, a gap 7 can be formed between the end section of the hook-shaped element and the base 1 (not shown in figures).

In another embodiment of the device, the locking devices 3 can be made elastically bendable (due to the use of materials with the required degree of elasticity), with the possibility of bending them to install a condom during manufacture and packaging, followed by elastic bending back (after termination of exposure), as well as with the formation of gaps 7 (spacing) between its end section and the end section of the gripping means.

The number of locking devices 3 may be different, depending on their shape and location, but ensuring uniform unwinding of the condom, i.e. the location of the condom ring in one plane.

Any polymeric material with the required performance properties (optimal flexibility, strength, cytotoxic parameters, etc.) can be used as the material of the base 1, as well as of the gripping means 2 and the locking devices 3.

The locking device 3 can be integral with the base.

The base 1 is provided with handles 8 for holding with fingers. The shape, size and number of handles 8 may be different. In the preferred embodiment, each part of the base 1 is provided with a handle 8. In this case, the handles 8 can be provided with rests 9 or grooving 10, or can be made of a curved shape (for example, bending upwards) in order to prevent fingers from slipping when stretching parts of the base in different directions.

The holder is used as follows.

After opening the individual packing (for example, traditional foil packing or any other), the holder with a condom is removed. In this case, the locking devices are in the closed position, and the condom is securely fixed. Further, the user, holding the handles, breaks the base (pulls in different directions or bends the base) and spreads parts of the base in different directions. In this case, the condom is stretched by the gripping means, which in the process of stretching change their planar configuration from rectangular to square (shown by a dash-double dotted line in FIGS. 1 and 5), thereby providing the largest area for stretching the condom and allowing the user to put it on quickly, without touching with fingers. As the condom is put on, it is smoothly and evenly unwound (rolled out) due to the action of the locking devices that simultaneously securely fix the condom ring in one plane, thereby excluding jumping off the gripping means. At the final stage of putting on, when the thickness of the condom ring is the smallest, parts of the base are easily separated, since the thin ring comes out through the gaps (spacing) between the end sections of the hook-shaped elements and the end sections of the gripping means.

Thus, the invention makes it possible to securely hold a condom when putting it on, as well as during transportation and storage in the individual packing.

1. A condom holder, including a base with means for gripping the condom ring, comprising locking devices.

2. The holder according to claim 1, wherein the locking devices are made in the form of latches with a stopper.

3. The holder according to claim 1, wherein the base is made detachable.

4. The holder according to claim 1, wherein the means for gripping the condom ring are hook-shaped.

5. The holder according to claim 1, wherein the base is provided with handles for holding with fingers.
